(19) United States
(12) Patent Application Publication
(10) Pub. No.: US 2024/0279615 A1
(43) Pub. Date: Aug. 22, 2024

(54) COMPOSITIONS COMPRISING DIPEPTIDES AND TRACE ELEMENTS

(71) Applicant: Evonik Operations GmbH, Essen (DE)

(72) Inventors: Martin Schilling, Bonn (DE); Anne Benedikt, Frankfurt (DE); Ann-Katrin Kreitmann, Ginsheim-Gustavsburg (DE); Christian Keßler, Aschaffenburg (DE); Mario Gomez, Darmstadt (DE); Stefan Randl, Frankfurt (DE)

(73) Assignee: Evonik Operations GmbH, Essen (DE)

(21) Appl. No.: 18/691,216

(22) PCT Filed: Aug. 24, 2022
(86) PCT No.: PCT/EP2022/073538

§ 371 Date: Mar. 12, 2024

(30) Foreign Application Priority Data
Sep. 15, 2021 (EP) 21196824.3

(51) Int. Cl. C12N 5/071; A61K 8/19; A61K 8/27; A61K 8/64; A61Q 19/00; C12N 5/0775

(52) U.S. Cl. CPC C12N 5/0682; A61K 8/19; A61K 8/27; A61K 8/64; A61Q 19/00; C12N 5/0662; A61K 2800/10; C12N 2500/20; C12N 2500/32

(57) ABSTRACT

Compositions that have dipeptides, trace metal ions, and a molar ratio between 10000 and 20 can be used in a cell culture medium. More specifically, improved culture media for use in biotechnological production processes, processes employing such improved media, and to products obtained from the processes using the improved culture media.

### FIELD OF THE INVENTION

The present invention relates to compositions comprising at least one dipeptide consisting of two amino acids, said amino acids being natural amino acids and at least one of said amino acid being cysteine (Cys) and at least one trace metal ion.

Moreover, the present invention relates to biotechnological production processes. More specifically, the present invention relates to improved culture media for use in biotechnological production processes, processes employing such improved media, and to products obtained from the processes using the improved culture media.

### BACKGROUND OF THE INVENTION

Chemically defined dipeptides are often used to replace difficult-to-formulate amino acids in cell culture media that are used in the production of biopharmaceuticals. The formation of a dipeptide increases the stability of glutamine and the solubility of tyrosine and cysteine. Well known examples are alanyl-glutamine, glycyl-glutamine, glycyl-tyrosine, alanyl-tyrosine and N, N′-di-alanyl-cysteine.

They are efficiently used as nutrients and their uptake and metabolism have been investigated in detail (Sánchez-Kopper et al. AMB Expr (2016) 6:48, Verhagen et al. Eng Life Sci. (2020); 1-11).

WO 2011/133902 discloses cell culture media comprising dipeptides, wherein the dipeptides include amino acids having a low solubility in water, in this case tyrosine and cysteine. Cysteine does not only have a low solubility in water, but it is also unstable, due to the presence of reactive thiol group. Furthermore Cysteine applied in higher concentrations such as 10 mM can decrease the cell viability. The authors have found that by incorporation of tyrosine and cysteine in dipeptides, solubility and stability problems of the amino acids can be ameliorated. The higher stability and solubility enables the formulation of chemically defined, highly concentrated media, that are required to run intensified and highly productive industrial cell culture processes.

Trace elements are micronutrients required by all animal and human cells to support metabolism. Examples of essential trace elements are iron, zinc and copper (Frieden, Journal of Chemical Education (1985), Vol 62, No 11, 917-923). Trace elements become toxic when provided in excess amounts and it can be difficult to provide enough but not too much of these micronutrients.

This is a well-known challenge when it comes to the formulation of cell culture media that are used to cultivate animal or human cells in vitro, e.g. in the context of biopharmaceutical, cell culture-based production of monoclonal antibodies; viral vaccines or cell therapies. Balancing sufficient supply against toxic effects can be difficult, especially when chemically defined, serum-free and even protein free media are required. It is further complicated by the fact that trace metals are often present as impurities of other media ingredients, such as amino acids.

The trace element concentrations defined in classical basal media recipes vary across a relatively wide concentration range between the different recipes. For example, concentrations of zinc and copper are in a range of low nanomolar to low micromolar but the largest part is traditionally introduced via sera, that contain between 10 and 30 μM of these trace elements (Vargas Arigony et al. BioMed Research International (2013) Article ID 597282).

When serum-free, chemically defined media are required to increase safety, consistency and performance in modern biopharmaceutical cell culture, these trace elements have to be added in a chemically defined form, which means inorganic salts.

There is limited published data on the trace metal content of chemically defined bioproduction media. However, it was shown that addition of sufficient amounts of copper and zinc is important to support growth, product formation and has a direct effect on primary metabolism, such as lactate consumption. It was also shown to have positive effects on product quality. The concentration effect was systematically investigated in a number of publications. For copper, concentrations between 0.05 to 100 μM are described (Yuk et al. Biotechnol. Prog. (2015), 31; 1: 226-238; Chaderjian et al. Biotechnol. Prog. (2005), 21:550-553). For zinc, concentrations between 3 and 150 μM are described (Roca et al. Cytotechnoloy (2019) 71:915-924; Graham et al. Applied Microbiology and Biotechnology (2020), 104: 1097-1108). In all cases, beneficial effects of addition could be demonstrated but high concentrations resulted in reduced performance or toxicity.

There is evidence in the literature that the toxic effects of trace metals are caused by redox reactions with other media components which leads to the formation of reactive oxygen species (ROS) that become toxic at higher concentrations (Keenan et al., In Vitro Cellular & Developmental Biology—Animal (2018) 54:555-558; Graham et al., Biotechnology and Bioengineering. 2019; 116:3446-3456).

The amino acid L-cysteine participates in redox reactions with metal ions and contributes to ROS formation. In these reactions, L-cysteine is oxidized to the disulfide form L-cysteine. This is described in the literature for Cu²⁺ ions in the presence of L-cysteine.

As both trace metals and sufficient amounts of a cysteine source a required in cell culture media to maximize productivity of the cell culture, there remains a need to improve cell culture media formulations to ensure sufficient trace metal supply without toxicity. There is also a need to provide sufficient cysteine equivalents while providing enough trace metals to cells. Therefore, it was a goal of the present invention to provide a cell culture medium providing a cysteine source and ensure supply with trade metals, but with reduced toxicity of both trace metals and cysteine.

### SUMMARY OF THE INVENTION

The above shortcomings are addressed by the present invention. The invention is defined by the terms of the appended independent claims. Preferred embodiments of the invention are defined by the dependent claims.

Surprisingly it was found that addition of dipeptides to cell culture media reduces the toxicity of metal ions. Moreover, it was found that L-cysteine induced metal toxicity could be reduced by addition of dipeptides. Finally, it was found that L-cysteine induced metal toxicity could be fully suppressed by replacing L-cysteine by cysteine dipeptides and that cellular viability was actually increased when compared to the respective controls.

The composition according to the present invention comprises at least one dipeptide consisting of two amino acids, said amino acids being natural amino acids and at least one of said amino acid being cysteine (Cys) and at least one trace metal ion, wherein the molar ratio of the dipeptide to the trace metal ion is between 10000 and 20.

The compositions according to the present invention can also be a component part of a cosmetic product, a nutritional supplement, a nutrient solution for clinical nutrition, or a cell or tissue culture medium (basal, feed or perfusion medium).

The invention further relates to the use of a culture medium of the invention for culturing cells, preferably plant cells, animal cells or mammalian cells.

Another aspect of the invention relates to a method of manufacturing a cell culture product comprising the steps of (i) providing a cell capable of producing said cell culture product; (ii) contacting said cell with a culture medium according to the invention; and (iii) obtaining said cell culture product from said culture medium or from said cell.

Preferred embodiments of the invention are described in further detail in the following detailed description of the invention.

### DETAILED DESCRIPTION OF THE INVENTION

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention, for instance “free” cysteine, is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The present invention generally relates to a composition comprising at least one dipeptide consisting of two amino acids, said amino acids being natural amino acids and at least one of said amino acid being cysteine (Cys) and at least one trace metal ion, wherein the molar ratio of the dipeptide to the trace metal ion is between 10000 and 20.

A “peptide” shall be understood as being a molecule comprising at least two amino acids covalently coupled to each other by alpha-peptide bonds (R¹—CO—NH—R²).

A “dipeptide” shall be understood as being a molecule comprising two amino acids covalently coupled to each other by an alpha-peptide-bond (R¹—CO—NH—R²).

The expression “Xxx”, when used herein in connection with an amino acid, shall be understood as referring to any natural amino acid as defined in the following.

An “amino acid”, in the context of the present invention, shall be understood as being a molecule comprising an amino functional group (—NH₂) and a carboxylic acid functional group (—COOH), along with a side-chain specific to the respective amino acid. In the context of the present invention, both alpha- and beta-amino acids are included. Preferred amino acids of the invention are alpha-amino acids, in particular the 20 “natural amino” acids including cysteine as follows:
Alanine (Ala/A)
Arginine (Arg/R)
Asparagine (Asn/N)
Aspartic acid (Asp/D)
Cysteine (Cys/C)
Cystine (Cyss/C2)
Glutamic acid (Glu/E)
Glutamine (Gln/Q)
Glycine (Gly/G)
Histidine (His/H)
Isoleucine (Ile/I)
Leucine (Leu/L)
Lysine (Lys/K)
Methionine (Met/M)
Phenylalanine (Phe/F)
Proline (Pro/P)
Serine (Ser/S)
Threonine (Thr/T)
Tryptophan (Trp/W)
Tyrosine (Tyr/Y)
Valine (Val/V)

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention (for instance “free cysteine”), is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The expression “N-acylated”, with reference to a chemical compound, such as an amino acid, shall be understood as meaning that the N-acylated compound is modified by the addition of an acyl group to a nitrogen functional group of said compound. Preferably, the acyl group is added to the alpha-amino group of the amino acid.

It is preferred, when the molar ratio of the dipeptide to the trace metal ion is between 5000 and 20, preferably between 1000 and 20.

In a preferred configuration, the trace metal is selected from iron, lithium, zinc, copper, chromium, nickel, cobalt, vanadium, molybdenum, manganese, the trace metal ion preferably being a copper ion. It is particularly preferred to use (Cu²⁺) ions.

In another preferred configuration, the dipeptide concentration is between 0.1 and 200 mM, preferable between 0.2 and 20 mM, most preferable between 0.5 and 10 mM and the trace metal ion concentration is between 0.1 and 400 μM, preferably between 0.2 and 100 μM, most preferable between 0.5 and 20 μM.

In another preferred configuration, the dipeptide is present in the culture medium at a concentration of at least 1 mM, preferably at least 10 mM, more preferably at least 50 mM, more preferably at least 100 mM. At such high concentrations, the composition according to the present invention provides the advantage that the cysteine-containing dipeptides stabilize cysteine against oxidative precipitation.

It is preferred, when the dipeptide is Xxx-Cys Cys-Xxx, wherein Xxx is a natural amino acid, the dipeptide preferably being Ala-Cys, Cys-Ala, Lys-Cys or Cys-Lys.

It is particularly preferred, when the dipeptide is Xxx-Cys or Cys-Xxx and is the oxidized and dimerized form, whereby the dimerized dipeptide is coupled via a disulfide bond.

In another preferred configuration, the composition further comprises free cysteine.

The compositions can be prepared by mixing defined molar ratios of the dipeptide with respective trace metals in the appropriate ratio to prepare powdered products or liquid stock solutions that can be added to cell culture media. Alternatively, the dipeptides can be added to a cell culture medium already containing respective trace metals. Optionally, compositions additionally containing cysteine can be prepared adding suitable amounts of cysteine to the composition or directly to the cell culture medium.

If the dipeptide is a Cys-dipeptide, the preferred molar ratio of the dipeptide bound cysteine (via disulfide bond) to the free cysteine is 10 or lower, preferably 4 or lower, more preferably 2 or lower, more preferable 1 or lower, more preferable 0.5 or lower, most preferable 0.2 or lower. The mixtures can be in the form of solids (crystalline powders, agglomerates, etc.) or aqueous solutions. In the case of aqueous solutions, cysteine is added in a concentration of at least 1 mM, preferable at least 10 mM, more preferable at least 50 mM and most preferable at least 100 mM and the dipeptide is added at the appropriate molar ratio described above.

The compositions can be prepared by mixing at least one dipeptide, with one amino acid being cysteine (Cys), and a cysteine source selected from free cysteine and optionally cysteine (Cys-Cys). The invention thus relates to a culture medium comprising the composition.

In the context of this invention, Cys-peptides forming a disulfide bond via oxidized cysteine residues, shall be described by (Xxx-Cys)₂. The peptides may also be present as salts or in hydrate form. Such disulfide bond mediated dimers of Cys-dipeptides, for instance (Xxx-Cys)₂, are still considered as dipeptides in the sense of the invention.

Preferably, the composition has a pH-value at 25° C. of at least 5 or preferred of at least 6.

In an advantageous configuration of the present invention, a molar ratio of the peptide-bound cysteine to free cysteine is between 0.1 and 10, preferably between 0.2 and 4 or lower, most preferable between 0.5 and 2 In a preferred embodiment, the dipeptides are either in a reduced state (=free thiol) or oxidized state (=disulfide bonded), preferably in an oxidized state

In an alternative embodiment, the composition comprises a mixed disulfide of the dipeptide and the cysteine source.

In a preferred embodiment of the present invention, the dipeptide further comprises one more natural amino acids with a solubility of at least >10 g/l at a pH range between pH 6 and pH 9 and is preferably selected from glycine (Gly), alanine (Ala), serine (Ser), proline (Pro), aspartic acid (Asp), glutamic acid (Glu), lysine (Lys) or arginine (Arg).

In preferred embodiments, the dipeptide is not N-acylated. N-acylation is known to improve heat stability of certain dipeptide; however, it has been found that N-acylated dipeptides may also lead to inferior viable cell density and viability.

The present invention is also directed to a cosmetic product, a nutritional supplement or nutrient solution for clinical nutrition comprising the composition according to the present invention.

The cosmetic product may be a shampoo, conditioner, lotion, cream, or other formulations used to treat skin or hair. Nutritional supplements may be in liquid form, such as syrups or shots, or in solid form, such as capsules, soft-gels, gummies. The compositions can also be part of nutrient solutions for clinical enteral or parenteral nutrition, e.g. part of an amino acid solution such as Aminoven (Fresenius Kabi).

Moreover, the present invention also refers to a cell or tissue culture medium.

Another subject of the present invention is directed to a cell or tissue culture medium comprising the composition according to the present invention, which further comprises at least one carbohydrate, at least one free amino acid, at least one inorganic salt, a buffering agent and/or at least one vitamin. In a particularly preferred embodiment, the culture medium comprises all of at least one carbohydrate, at least one free amino acid, at least one inorganic salt, a buffering agent and at least one vitamin.

In one embodiment of the invention, the culture medium does not contain a growth factor. In accordance with this embodiment, the dipeptide of the invention may be used instead of a growth factor for promoting growth and/or proliferation of the cells in culture. In another embodiment of the invention, the culture medium does not contain any lipids.

According to another embodiment of the invention, the culture medium is in liquid form, in form of a gel, a powder, a granulate, a pellet or in form of a tablet.

In preferred embodiments, the culture medium of the invention is a defined medium, or a serum-free medium. For example, the compositions of the invention may be supplemented to the CHOMACS CD medium of Miltenyi Biotech (Bergisch Gladbach, Germany), to the PowerCHO-2 CD medium available from LONZA (Basel, Switzerland), the Acti-CHO P medium of PAA (PAA Laboratories, Pasching, Austria), the Ex-Cell CD CHO medium available from SAFC, the SFM4CHO medium and the CDM4CHO medium of ThermoFisher (Waltham, USA). The dipeptides of the invention may also be supplemented to DMEM medium (Life Technologies Corp., Carlsbad, USA). The invention, however, is not limited to supplementation of the above media.

In other preferred embodiments, the culture medium is a liquid medium in 2-fold, 3-fold, 3.33-fold, 4-fold, 5-fold or 10-fold concentrated form (volume/volume), relative to the concentration of said medium in use. This allows preparation of a “ready-to-use” culture medium by simple dilution of the concentrated medium with the respective volume of sterile water. Such concentrated forms of the medium of the invention may also be used by addition of the same to a culture, e.g., in a fed-batch cultivation or perfusion process.

The cell culture medium (cell or tissue culture basal, feed or perfusion medium) of the present invention may preferably contain all nutrients required for sustained growth and product formation. Recipes for preparing culture media, in particular cell culture media, are well known to the person skilled in the art (see, e.g., Cell Culture Technology for Pharmaceutical and Cell-Based Therapies, Öztürk and Wei-Shou Hu eds., Taylor and Francis Group 2006). Various culture media are commercially available from various sources.

The culture media of the invention may preferably include a carbohydrate source. The main carbohydrate used in cell culture media is glucose, routinely supplemented at 5 to 25 mM. In addition, any hexose, such as galactose, fructose, or mannose or a combination may be used.

The culture medium typically may also include at least the essential amino acids (i.e., His, Ile, Leu, Lys, Met, Phe, Thr, Try, Val) as well as non-essential amino acids. A non-essential amino acid is typically included in the cell culture medium if the cell line is not capable of synthesizing the amino acid or if the cell line cannot produce sufficient quantities of the amino acid to support maximal growth. In addition, mammalian cells can also use glutamine as a major energy source. Glutamine is often included at higher concentrations than other amino acids (2-8 mM). However, as noted above, glutamine can spontaneously break down to form ammonia and certain cell lines produce ammonia faster, which is toxic.

The culture media of the invention may preferably comprise salts. Salts are added to the cell culture medium to maintain isotonic conditions and prevent osmotic imbalances. The osmolality of a culture medium of the invention is about 300 mOsm/kg, although many cell lines can tolerate an approximately 10 percent variation of this value or higher. The osmolality of some insect cell cultures tends to be higher than 300 mOsm/kg, and this may be 0.5 percent, 1 percent, 2 to 5 percent, 5-10 percent, 10-15 percent, 15-20 percent, 20-25 percent, 25-30 percent higher than 300 mOsm/kg. The most commonly used salts in cell culture medium include Na⁺, K⁺, Mg²⁺, Ca²⁺, Cl⁻, SO₄²⁻, PO₄³⁻, and HCO₃⁻ (e.g., CaCl₂), KCl, NaCl, NaHCO₃, Na₂HPO₄).

Other inorganic elements (including further trace elements) may be present in the culture medium. They include Mn, Cu, Zn, Mo, Va, Se, Fe, Ca, Mg, Si, and Ni. Many of these elements are involved in enzymatic activity. They may be provided in the form of salts such as CaCl₂), Fe(NO₃)₃, MgCl₂, MgSO₄, MnCl₂, NaCl, NaHCO₃, Na₂HPO₄, and ions of the trace elements, such as, selenium, vanadium and zinc. These inorganic salts and trace elements may be obtained commercially, for example from Sigma (Saint Louis, Missouri).

The culture media of the invention preferably comprise vitamins. Vitamins are typically used by cells as cofactors. The vitamin requirements of each cell line vary greatly, although generally extra vitamins are needed if the cell culture medium contains little or no serum or if the cells are grown at high density. Exemplary vitamins preferably present in culture media of the invention include biotin, choline chloride, folic acid, i-inositol, nicotinamide, D-Ca⁺⁺-pantothenate, pyridoxal, riboflavin, thiamine, pyridoxine, niacinamide, A, B₆, B₁₂, C, D₃, E, K, and p-aminobenzoic acid (PABA).

Culture media of the invention may also comprise serum. Serum is the supernatant of clotted blood. Serum components include attachment factors, micronutrients (e.g., trace elements), growth factors (e.g., hormones, proteases), and protective elements (e.g., antitoxins, antioxidants, antiproteases). Serum is available from a variety of animal sources including human, bovine or equine serum. When included in cell culture medium according to the invention, serum is typically added at a concentration of 5-10% (vol.). Preferred cell culture media are serum-free.

To promote cell growth in the absence or serum or in serum reduced media, one or more of the following polypeptides can be added to a cell culture medium of the invention: for example, fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, any cytokine, such as interleukins 1, 2, 6, granulocyte stimulating factor, leukocyte inhibitory factor (LIF), etc.

In other embodiments, the cell culture medium does not comprise polypeptides (i.e., peptides with more than 20 amino acids).

One or more lipids can also be added to a cell culture medium of the invention, such as linoleic acid, linolenic acid, arachidonic acid, palmitoleic acid, oleic acid, polyenoic acid, and/or fatty acids of 12, 14, 16, 18, 20, or 24 carbon atoms, each carbon atom branched or unbranched), phospholipids, lecithin (phosphatidylcholine), and cholesterol. One or more of these lipids can be included as supplements in serum-free media. Phosphatidic acid and lysophosphatidic acid stimulate the growth of certain anchorage-dependent cells, such as MDCK, mouse epithelial, and other kidney cell lines, while phosphatidylcholine, phosphatidylethanolamine, and phosphatidylinositol stimulate the growth of human fibroblasts in serum-free media. Ethanolamine and cholesterol have also been shown to promote the growth of certain cell lines. In certain embodiment, the cell culture medium does not contain a lipid.

One or more carrier proteins, such as bovine serum albumin (BSA) or transferrin, can also be added to the cell culture medium. Carrier proteins can help in the transport of certain nutrients or trace elements. BSA is typically used as a carrier of lipids, such as linoleic and oleic acids, which are insoluble in aqueous solution. In addition, BSA can also serve as a carrier for certain metals, such as Fe, Cu, and Ni. In protein-free formulations, non-animal derived substitutes for BSA, such as cyclodextrin, can be used as lipid carriers.

One or more attachment proteins, such as fibronectin, laminin, and pronectin, can also be added to a cell culture medium to help promote the attachment of anchorage-dependent cells to a substrate.

The cell culture medium can optionally include one or more buffering agents. Suitable buffering agents include, but are not limited to, N-[2-hydroxyethyl]-piperazine-N′-[2-ethanesulfonic acid] (HEPES), MOPS, MES, phosphate, bicarbonate and other buffering agents suitable for use in cell culture applications. A suitable buffering agent is one that provides buffering capacity without substantial cytotoxicity to the cells cultured. The selection of suitable buffering agents is within the ambit of ordinary skill in the art of cell culture.

Polyanionic or polycationic compounds may be added to the culture medium to prevent the cells from clumping and to promote growth of the cells in suspension.

In a preferred embodiment, the culture medium is in liquid form. The culture medium, however, can also be a solid medium, such as a gel-like medium, e.g. an agar-agar-, carrageen- or gelatin-containing medium (powders, aggregated powders, instantized powders etc.). Preferably, the culture medium is in sterile form.

The culture medium of the present invention can be in concentrated form. It may be, e.g., in 2- to 100-fold concentrated form, preferably in 2-fold, 3-fold, 3.33-fold, 4-fold, 5-fold, 10-fold, 20-fold, 50-fold or 100-fold (relative to a concentration that supports growth and product formation of the cells). Such concentrated culture media are helpful for preparing the culture medium for use by dilution of the concentrated culture medium with an aqueous solvent, such as water. Such concentrated culture media may be used in batch culture but are also advantageously used in fed-batch or continuous cultures, in which a concentrated nutrient composition is added to an ongoing cultivation of cells, e.g., to replenish nutrients consumed by the cells during culture.

In other embodiments of the invention, the culture medium is in dry form, e.g., in form of a dry powder, or in form of granules, or in form of pellets, or in form of tablets.

The present invention also relates to the use of a culture medium of the invention for culturing cells. Another aspect of the invention relates to the use of a culture medium of the invention for producing a cell culture product.

A preferred embodiment of the invention relates to the use of a culture medium according to the invention for culturing animal cells or plant cells, most preferred mammalian cells. In specific embodiments the cells to be cultured are CHO cells, COS cells, VERO cells, BHK cells, HEK cells, HELA cells, AE-1 cells, NS0 cells, insect cells, fibroblast cells, muscle cells, nerve cells, stem cells, skin cells, endothelial cells and hybridoma cells. Preferred cells of the invention are CHO cells and hybridoma cells. Most preferred cells of the invention are CHO cells. Particularly preferred CHO cells of the invention are CHO DG44 and CHO DP12 cells.

Also included in the scope of the present invention is a method of culturing cells, said method comprising contacting said cells with a cell culture medium according to the invention. In one embodiment of the invention, the method of culturing cells comprises contacting the cell with a basal culture medium under conditions supporting the cultivation of the cell and supplementing the basal cell culture medium with a concentrated medium according to the present invention. In preferred embodiments, the basal culture medium is supplemented with the concentrated feed or medium on more than one day.

Another aspect of the invention relates to a method of producing a culture medium according to the invention, wherein said culture medium comprises a composition according to the invention. Methods of producing a culture medium according to the invention comprise at least one step of adding the composition of the invention to the culture medium. Likewise, an aspect of the invention relates to the use of a composition of the invention for producing a cell culture medium.

Another aspect of the invention relates to a method of modifying a culture medium, wherein said modifying of said culture medium comprises addition of the composition of the invention to said culture medium.

Another aspect of the invention relates to a method of producing a liquid culture medium, said method comprising providing solid medium according to the invention, e.g., in form of a dry powder, or in form of granules, or in form of pellets, or in form of tablets; and dissolving said solid culture medium in an aqueous medium, such as water.

Another aspect of the invention relates to the use of a composition according to the invention in a culture medium for culturing cells. Another aspect of the invention relates to the use of a composition according to the invention for cell culture.

The invention also relates to methods of manufacturing a cell culture product comprising the steps of (i) providing a cell capable of producing said cell culture product; (ii) contacting said cell with a culture medium of the invention; and (iii) obtaining said cell culture product from said culture medium or from said cell. Likewise, the present invention relates to the use of a composition according to the invention for manufacturing a cell culture product.

In preferred methods, the cell culture product is a therapeutic protein, a diagnostic protein, a polysaccharide, such as heparin, an antibody, a monoclonal antibody, a growth factor, an interleukin, virus, virus-like particle or an enzyme

Cultivation of cells, according to the invention can be performed in batch culture, in fed-batch culture or in continuous culture.

### EXAMPLES

### Materials:

TABLE 1
Materials used for in vitro cytotoxicity assay

| Material | Supplier |
|---|---|
| Human bone marrow stromal cells | Stemcell, Vancouver (Canada) |
| (Cat. No 70071) | |
| MesenCult ™-ACF Plus Culture Kit | Stemcell, Vancouver (Canada) |
| Agarabi CHO (ATCC CRL-3440) | American Type Culture Collection, |
| | Manassas (USA) |
| ActiPro ™ medium | Cytiva, Marlborough (USA) |
| Gentamicin (50 mg/mL) | Thermo Fisher Scientific, |
| | Massachusetts (USA) |
| L-Glutamine | Stemcell, Vancouver (Canada) |
| Copper (II) sulfate pentahydrate | Sigma-Aldrich, Missouri (USA) |
| (CuSO₄) | |
| L-Cysteine | Sigma-Aldrich, Missouri (USA) |
| (N,N′-di-L-alanyl-L-cystine) | Evonik Operations GmbH, |
| | Darmstadt (Germany) |
| (N,N′-di-L-lysyl-L-cystine) | Evonik Operations GmbH, |
| | Darmstadt (Germany) |
| Cell culture microplate, | Greiner Bio-One, |
| 96 well, white | Kremsmünster (Austria) |
| CellTiter-Glo Luminescent Cell | Promega GmbH, Madison (USA) |
| Viability Assay (Cat. No G7570) | |

TABLE 2
Devices used for in vitro cytotoxicity assay.

| Device | Supplier |
|---|---|
| Safety cabinet HERA | Thermo Fisher Scientific GmbH, |
| Safe 2020 | Dreieich (Germany) |
| Heracell ™ 150i CO₂ | Thermo Fisher Scientific GmbH, |
| Incubator | Dreieich (Germany) |
| Automatic cell | Thermo Fisher Scientific GmbH, |
| counter Countess | Dreieich (Germany) |
| Centrifuge 5415R | Eppendorf, Hamburg (Germany) |
| Vortex-Genie 2 | Scientific Industries, |
| | Bohemia (USA) |
| TECAN Infinite | Tecan Group Ltd., |
| 200 Pro | Männedorf (Switzerland) |

### Methods:

### In Vitro Cytotoxicity Assay

Human mesenchymal stem cells (MSCs) were cultivated in MesenCult™-ACF Plus Culture Kit (Stemcell Technologies). The medium was prepared according to the manufacturer's instructions with addition of 30 mg/mL gentamicin and 2 mM I-glutamine. Agarabi CHO cells were cultivated in ActiPro medium supplemented with 6 mM I-glutamine. “Complete growth medium” references the medium with added supplements used for the cultivation of the corresponding cell type.

Stock solutions of Cu(II), I-cysteine, N, N′-di-I-alanyl-I-cysteine ((Ala-Cys)₂) and N, N′-di-I-lysyl-I-cysteine ((Lys-Cys)₂) were brought into solution in the corresponding complete growth medium immediately prior to their use. For the assay performed with combinations of Cu(II) and I-cysteine, (Ala-Cys)₂ or (Lys-Cys)₂ on MSCs, the dissolved single compounds were mixed together at 2× treatment concentration. On MSCs, I-cysteine and the dipeptides were tested at 0.5 mM, 1 mM and 5 mM; Cu(II) was tested at 0.2 μM, 1 μM and 5 μM. For the treatment of Agarabi CHO cells, serial dilutions of I-cysteine, (Ala-Cys)₂ and (Lys-Cys)₂ stock solutions in complete growth medium were performed with 2× treatment concentration at each dilution step. On Agarabi CHO cells, I-cysteine and the dipeptides were tested at 2.5 mM, 5 mM and 10 mM; Cu(II) was tested at 2.5 μM, 10 μM and 40 μM.

The cytotoxicity of the test compounds and their combinations was assayed on MSCs and Agarabi CHO cells cultivated in white 96-well cell culture plates at 5.000 cells/well. MSCs (50 μL/well) were cultivated for 24 h in a CO₂-incubator (37° C., 5% CO₂, 95% humidity) to ensure cell adhesion. This was followed by the addition of the prepared 2× concentrated test compounds and mixtures (50 μL/well). The plate was incubated for 24 h (37° C., 5% CO₂, 95% humidity). Single compounds as well as MSCs cultured in complete growth medium without any additional compounds were used as controls. Each condition was tested in triplicates. Agarabi CHO cells were seeded (50 μL/well) using complete growth medium as well as complete growth medium containing 2× concentrated Cu(II) test concentrations. This was followed immediately by the addition of 50 μL of the prepared serial dilution of l-cysteine, (Ala-Cys)₂ and (Lys-Cys)₂. The Agarabi CHO cells were incubated for 96 h (37° C., 5% CO₂, 95% humidity). All single components on Agarabi CHO cells (“single component controls”), Agarabi CHO cells in complete growth medium without the addition of any test compounds (“untreated cells”) and complete growth medium with and without the combination of Cu(II) with I-cysteine, (Ala-Cys)₂ or (Lys-Cys)₂ (“media blanks”) were used as controls. For the controls, the compounds were used at the highest concentration tested. Four replicates of each condition were tested.

After incubation, the viability of the treated cells was determined based on adenosine triphosphate (ATP) quantification using the CellTiter-Glo® Luminescent Cell Viability Assay (Promega) according to the manufacturer's instructions.

To calculate the cell viability, resulting luminescence values were averaged and normalized to the signal emitted by the untreated cells.

### Example 1: Replacement of Cysteine by Dipeptides Abolished Negative Effects of Trace Metals and Even Contributes to Increased Viability

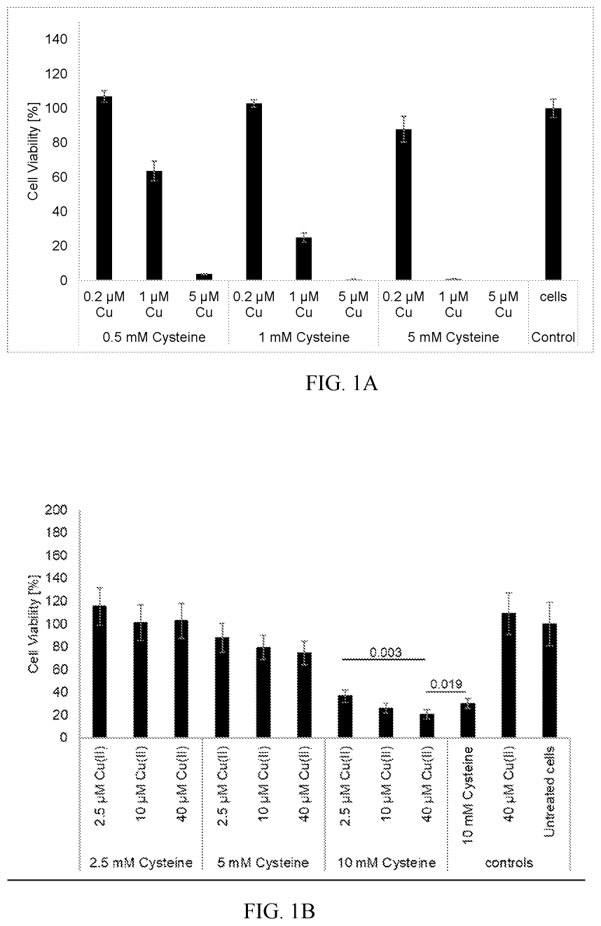

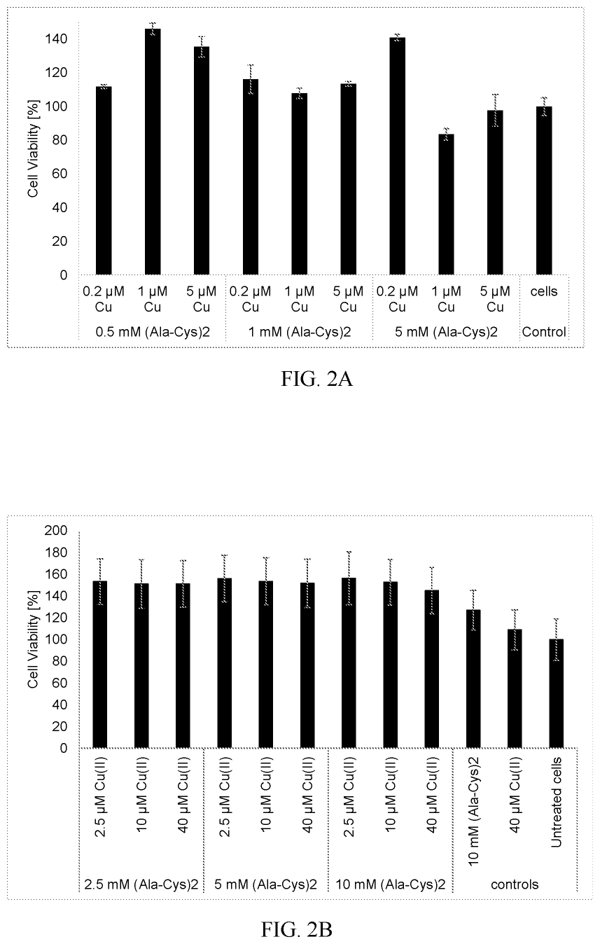

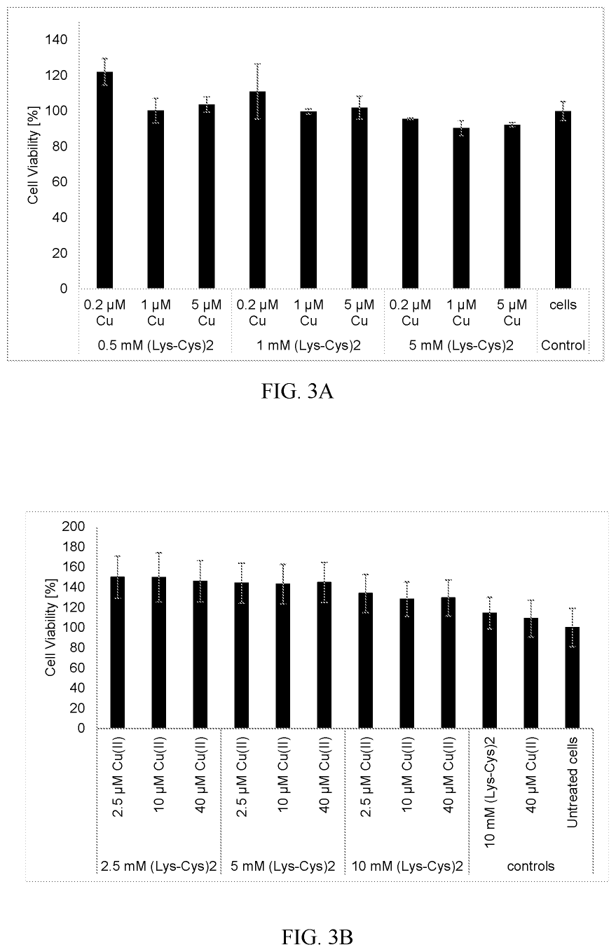

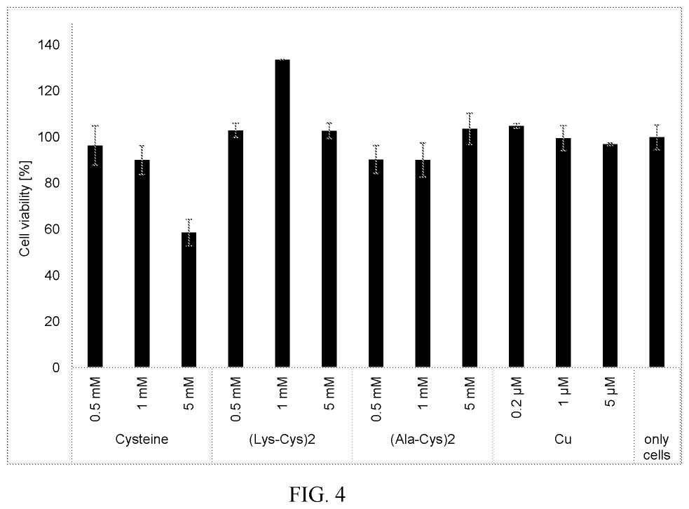

The addition of L-cysteine and Cu(II) at several concentration to cell culture media resulted in a significant, concentration depended reduction in cell viability (FIGS. 1A and B). No toxic effect was observed replacing L-cysteine with an equimolar amount of the dipeptides (Ala-Cys)₂ and (Lys-Cys)₂. Instead, viability was even increased providing beneficial effects of combining dipeptides with Cu(II) (FIGS. 2A and B, FIGS. 3A and B). This open ups the possibility to increase the concentration of Cu(II) as well as the L-cysteine source concentration in cell culture media using dipeptides instead of L-cysteine. The addition of individual compounds in the respective concentrations were investigated as controls. Toxicity at highest concentration tested was only observed with L-cysteine (FIG. 4).

FIGS. 1-A and 1-B shows the effects of Cu(II) and L-cysteine addition at different concentrations and ratios on the viability of MSCs and CHO cells, respectively. A strong increase in toxicity was observed with increasing concentrations of Cu as well as L-cysteine.

FIGS. 2-A and 2-B shows the effects of Cu(II) and (Ala-Cys)₂ addition at different concentrations and ratios on the viability of MSCs and CHO cells, respectively. In contrast to FIGS. 1-A and 1-B, no toxic effects were observed. For selected combinations of (Ala-Cys)₂ and Cu(II), a pronounced positive effect on viability was observed.

FIGS. 3-A and 3-B shows the effects of Cu(II) and (Ala-Cys)₂ addition at different concentrations and ratios on the viability of MSCs and CHO cells, respectively. In contrast to FIGS. 1-A and 1-B, no toxic effects were observed. For selected combinations of (Lys-Cys)₂ and Cu(II), a pronounced positive effect on viability was observed.

FIG. 4 shows control experiments depicting the effect of the addition of single components (L-cysteine, (Ala-Cys)₂, (Lys-Cys)₂, Cu(II)) at various concentrations on the viability of MSCs. Only L-cysteine negatively affected viability. Cu(II) up to 5 μM (MSCs) or 40 μM (CHO cells) did not have a negative effect on viability.

Summarizing, it was surprisingly found that Cu(II) catalyzed L-cysteine toxicity can be reduced by the addition of peptides and when L-cysteine is replaced by Cys-peptides. Higher concentrations of trace metals can thus be safely provided to cells and viability can even be increased.

1. A composition, comprising:
at least one dipeptide consisting of two amino acids, said amino acids being natural amino acids and at least one of the said amino acids being cysteine (Cys), and
at least one trace metal ion,
wherein a molar ratio of the at least one dipeptide to the at least one trace metal ion is between 10000 and 20.

2. The composition according to claim 1, wherein the molar ratio of the at least one dipeptide to the at least one trace metal ion is between 5000 and 20.

3. The composition according to claim 1, wherein the at least one trace metal ion is at least one selected from the group consisting of iron, lithium, zinc, copper, chromium, nickel, cobalt, vanadium, molybdenum, and manganese.

4. The composition according to claim 1, wherein the at least one dipeptide concentration is between 0.1 and 200 mM and the at least one trace metal ion concentration is between 0.1 and 400 μM.

5. The composition according to claim 1, wherein the at least one dipeptide is Xxx-Cys or Cys-Xxx, wherein Xxx is a natural amino acid, the dipeptide being at least one selected from the group consisting of Ala-Cys, Cys-Ala, Lys-Cys, and Cys-Lys.

6. The composition according to claim 5, wherein the at least one dipeptide is in the oxidized and dimerized form.

7. The composition according to claim 1, further comprising free cysteine.

8. The composition according to claim 7, wherein a molar ratio of the dipeptide bound cysteine to the free cysteine is from about 0.1 to 10.

9. A cosmetic product, nutritional supplement, or nutrient solution for clinical nutrition, comprising:
the composition according to claim 1.

10. A cell culture medium, comprising:
a composition according to claim 1, said culture medium further comprising at least one carbohydrate, and/or at least one additional free amino acid, and/or at least one inorganic salt, and/or a buffering agent, and/or at least one vitamin.

11. The culture medium according to claim 10, wherein said culture medium is in liquid form, in form of a gel, a powder, a granulate, a pellet, or in the form of a tablet.

12. The culture medium according to claim 10, wherein said culture medium is in a 2- to 100-fold concentrated form, relative to the concentration of the culture medium in use.

13. A method for culturing cells, the method comprising:
culturing cells in a culture medium according to claim 10.

14. The method of claim 13, wherein said cells are selected from the list consisting of CHO cells, COS cells, VERO cells, BHK cells, HEK cells, HELA cells, AE-1 cells, NS0 cells, insect cells, fibroblast cells, muscle cells, nerve cells, stem cells, skin cells, endothelial cells, immune cells, and hybridoma cells.

15. A method of manufacturing a cell culture product, the method comprising:
providing a cell capable of producing said cell culture product;
contacting said cell with a culture medium of claim 10; and
obtaining said cell culture product from said culture medium or from said cell.

16. The composition according to claim 1, wherein the molar ratio of the at least one dipeptide to the at least one trace metal ion is between 1000 and 20.

17. The composition according to claim 1, wherein the at least one trace metal ion is a copper ion.

18. The composition according to claim 1, wherein the at least one dipeptide concentration is between 0.5 and 10 mM and the at least one trace metal ion concentration 0.5 and 20 μM.

19. The composition according to claim 1, wherein a molar ratio of the dipeptide bound cysteine to the free cysteine is from about 0.5 to 2.

20. The culture medium according to claim 10, wherein said culture medium is in a 2-fold, 3-fold, 3.33-fold, 4-fold, 5-fold or 10-fold concentrated form, relative to the concentration of the culture medium in use.
